(19) United States
(12) Patent Application Publication
(10) Pub. No.: US 2005/0220837 A1
(43) Pub. Date: Oct. 6, 2005

(54) Antimicrobial hyaluronic acid coatings for orthopedic implants

(76) Inventors: John Arthur Disegi, Reading, PA (US); Robert Geoffrey Richards, Davos Dorf (CH); Llinos Gwawr Harris, Davos Platz (CH)

Correspondence Address: JONES DAY, 222 EAST 41ST ST, NEW YORK, NY, 10017, US

(21) Appl. No.: 10/955,777

(22) Filed: Sep. 30, 2004

Related U.S. Application Data
(60) Provisional application No. 60/506,760, filed on Sep. 30, 2003.

(51) Int. Cl. A61F 2/28; A61F 13/00

(52) U.S. Cl. 424/423; 623/16.11

(57) ABSTRACT

The invention relates to an implant wherein the surface of the implant is coated with hyaluronic acid or a derivative thereof. The coated implants resist microbial growth.

This application claims the benefit of U.S. Provisional Patent Application No. 60/506,760, filed Sep. 30, 2003, the entire disclosure of which is incorporated herein by reference.

### FIELD OF THE INVENTION

The invention relates to an implant wherein the surface of the implant is coated with hyaluronic acid or a derivative thereof. The coated implants resist microbial growth.

### BACKGROUND OF THE INVENTION

Once biomaterial implants are implanted in a body they are coated thereafter with host plasma constituents, including protein components of the extracellular matrix (ECM) such as fibrin; and eventually host cells—leading to the formation of soft and hard tissue (see Baier et al., J. Biomed. Mater. Res. 18:337-355 (1984)). The ability of Staphylococcus aureus (S. aureus) and Staphylococcus epidermidis (S. epedermis) to adhere to the extracellular matrix and plasma proteins deposited on biomaterials is a significant factor in the pathogenesis of orthopaedic-device related infections, and the resultant bacteria is reported to form Biofilms (see Hoyle et al., Prog. Drug Res. 37:91-105 (1991)).

Biofilm formation is a two-step process that requires the adhesion of bacteria to a surface followed by cell-cell adhesion, forming multiple layers of the bacteria (see, e.g., Cramton et. al., Infect. Immun. 67: 5427-5433 (1999)). Once a biofilm has formed, it is difficult to clinically treat because the bacteria in the interior of the biofilm are protected from both phagocytosis and antibiotics (see Hoyle). Over the last decade, systemic antibiotics have not provided an effective treatment against infections associated with implants (see, e.g., Petty et. al., J. Bone Joint Surg. 67: 1236-1244 (1985); Barth et al., Biomat. 10: 325-328 (1989); Wassall et al, J. Biomed. Mater. Res. 36(3): 325-330 (1997); and Lowy, N. Engl. J. Med. 339(8): 520-32 (1988)).

Hallab et al., Tissue Eng. 7(1):55-71 (2001); and Lange et al., Biomol. Eng. 19:255-61 (2002) report that surface properties of medical implants, including topography and chemistry are important in the promotion or inhibition of cell and bacterial adhesion. Hence, various studies have modified implant surfaces in an attempt to decrease infections (see, e.g., Koerner et al., Biomaterials 23(14): 2835-40 (2002); Park et al., Biomaterials 19: 851-9 (1998); and Lowy, N. Engl. J. Med. 339(8): 520-32 (1988)).

The adherence of eukaryotic cells and ECM proteins to modified surfaces has received much more attention than bacterial adherence (see Anselme et al., J. Biomed. Mater. Res. 49(2): 155-66 (2002); and Lowy, N. Engl. J. Med. 339(8): 520-32 (1988); and Hallab et al., Tissue Eng. 7(1):55-71 (2001)). Different surface treatments have been used to modify the topography and surface chemistry of materials such as titanium (see, e.g., Puleo et al., Biomaterials 20: 2311-21 (1999); Lowey; and Hallab). The Lowey article describes one approach where the surface is polished. Another approach is to coat the surface with an antimicrobial or protein resistant coating (see, e.g., Koerner; Nagaoka et al., ASAIO J. 141(3): M365-8 (1995); and Xiao in Titanium in Medicine 417-449 (Springer-Verlag, Heidelberg and Berlin, 2001)).

Hydrophilic coatings, such as hyaluronan, are reported to have anti-adhesive properties. For example, Pavesio et al., Med. Device Technol. 8(7): 20-1 and 24-7 (1997) and Cassinelli et al., J. Biomater. Sci. Polym. Ed. 11(9): 961-77 (2000) describe coated polymeric medical devices (e.g., intraocular lenses, stents and catheters) with decreased fibroblast and Staphylococcus epidermidis adhesion.

U.S. Pat. No. 4,500,676 to Balazs et al. describes polymeric materials and articles made therefrom that are rendered biocompatible by including hyaluronic acid or a salt thereof with the polymeric material

U.S. Pat. No. 4,853,225 to Wahlig et al. describes an implantable medicament depot containing physiologically acceptable excipients and at least one delayed release active compound which is a chemotherapeutic of the gyrase inhibitor type.

U.S. Pat. No. 5,166,331 to della Valle et al., U.S. Pat. No. 5,442,053 to della Valle et al., and U.S. Pat. No. 5,631,241 to della Valle et al. all describe pharmaceutically useful fractions of hyaluronic acid for various applications, i.e., between 50,000 and 100,000 Daltons which is useful for wound healing, and between 500,000 and 730,000 Daltons which is useful for intraocular and intraarticular injections. The hyaluronic acid in these references may be present as free acid, as an alkali or alkaline earth metal salt, or as a salt with one or more pharmacologically active substances.

U.S. Pat. No. 5,505,945 to Gristina et al., U.S. Pat. No. 5,530,102 to Gristina et al., U.S. Pat. No. 5,707,627 to Gristina et al., and U.S. Pat. No. 5,718,899 to Gristina et al., as well as International Publication No. WO 94/15640, all describe compositions containing a high concentration of immunoglobulins IgA, IgG, and IgM to combat infections from microorganisms and viruses. The immunoglobulins in these references can be immobilized on a variety of biocompatible materials such as collagen, fibrin, hyaluronan, biodegradable polymers, and fragments thereof.

U.S. Pat. No. 5,929,048 to Falk et al., U.S. Pat. No. 5,985,850 to Falk et al., and U.S. Pat. No. 6,069,135 to Falk et al. all describe compositions, dosages, and methods for treating underperfused and pathological tissues containing a therapeutic amount of hyaluronic acid and/or a salt thereof and/or homologues, analogues, derivatives, complexes, esters, fragments, and subunits thereof.

U.S. Pat. No. 6,428,579 to Valentini describes a coated implantable device having a gold layer on the surface to which bioactive molecules are attached through a gold-sulfhydryl bond.

U.S. Pat. No. 6,503,556 to Harish et al. describes methods of forming a coating on an implantable device or endoluminal prosthesis. The coating in this reference can also be used for the delivery of an active ingredient, radioopaque elements, or radioactive isotopes.

U.S. Pat. No. 6,617,142 to Keogh et al. describes methods for forming a coating of an immobilized biomolecule on a surface of a medical device to impart improved biocompatibility for contacting tissue and bodily fluids.

U.S. Patent Publication No. 2003/0091609 A1 and International Publication No. WO 02/058752 both describe a medical device, as well as a method of making and using the same, containing a carrier (i.e., a polymer) and a polynucleotide or a cell that expresses an antimicrobial polynucleotide.

There is a need, however, for improved implants that resist microbial growth.

### SUMMARY OF THE INVENTION

The invention relates to implants coated with hyaluronic acid or a derivative thereof. The coated implants resist microbial growth.

In one embodiment, the invention is directed an implant coated with hyaluronic acid or a derivative thereof, wherein the implant is a metal, a metal alloy, a ceramic, or a combination thereof.

In another embodiment, the invention is directed an implant coated with hyaluronic acid or a derivative thereof, wherein the implant is substantially free of a plastic or polymer.

In another embodiment, the invention is directed to an orthopedic implant coated with hyaluronic acid or a derivative thereof.

In another embodiment, the invention is directed an implant coated with a coating comprising: (a) hyaluronic acid or a derivative thereof; and (b) and an antimicrobial agent.

In another embodiment, the invention relates to a multi-coated implant comprising: (a) a first layer residing on the surface of the implant; and (b) a second layer comprising hyaluronic acid or a derivative thereof residing on the first layer.

The present invention can be understood more fully by reference to the following figures, detailed description and examples, which are intended to exemplify non-limiting embodiments of the invention.

### BRIEF DESCRIPTION OF THE DRAWINGS

FIGS. 1A-1F show the field emission scanning electron microscope (FESEM) images of S. aureus adhered to standard titanium surfaces and coated surfaces after 1 hour culturing (TS, TSS, THY, TIG, TLF, TAST) and show that very few bacteria are seen on the THY surface compared to the other surfaces.

FIGS. 2A-2B show the number density of S. aureus adhering to the different surfaces. (a) Standard titanium (TS and TSS) and standard titanium coatings, (b) Standard titanium (TS) and polished surfaces.

FIGS. 3A-3B show fluorescence microscopy images of S. aureus adhering to standard titanium (TS) and chemically polished titanium (TC). The white dots represent the live bacteria, which were seen red in the original images.

FIG. 4 shows SEM images of S. epidermidis on CA, CAC, CP and CPC surfaces after culturing for 48 h.

FIG. 5 shows SEM images of hTERT fibroblast cells, after 48 h (left images) and 96 h (right images) of culturing on CA and CP surfaces.

FIG. 6 shows SEM images of hTERT fibroblast cells, after 48 h (left images) and 96 h (right images) of culturing on CAC and CPC surfaces .

FIG. 7 shows SEM images of hTERT fibroblast cells, after 48 h (left images) and 96 h (right images) of culturing on CC, CH, CHP, and CHR surfaces.

FIG. 8 shows SEM images of hTERT fibroblast cells after 48 h (left image) and 96 h (right image) of culturing on a CHC surface.

### DETAILED DESCRIPTION OF THE INVENTION

As noted above, the invention is directed to an implant coated with hyaluronic acid or a derivative thereof (the “Antimicrobial Coating”). The coated implant resists microbial growth. Examples of microbial growth that can be resisted include, but are not limited to, Staphlococcus aureus and Staphlococcus epidermidis.

The coated implants of the invention can be bioabsorbable, resorbable, or permanent. The implants of the invention can be used in osseointegrative, osteosynthetic, orthopedic, and dental applications. Representative implants include, but are not limited to, void fillers (e.g., bone void fillers), adjuncts to bone fracture stabilization, intramedullary fixation devices, joint augmentation/replacement devices, bone fixation plates (e.g., craniofacial, maxillofacial, orthopedic, skeletal, and the like), screws, tacks, clips, staples, nails, pins, rods, anchors (e.g., for suture, bone, or the like), scaffolds, stents, meshes (e.g., rigid, expandable, woven, knitted, weaved, etc.), sponges, implants for cell encapsulation or tissue engineering, drug delivery devices (e.g., antivirals; antibiotics; carriers; bone ingrowth induction catalysts such as bone morphogenetic proteins, growth factors, peptides, and the like.), monofilament or multifilament structures, sheets, coatings, membranes (e.g., porous, microporous, and resorbable membranes), foams (e.g., open cell and closed cell foams), screw augmentation devices, cranial reconstruction devices, a heart valve, and pacer lead.

In one embodiment, the implant is an orthopedic implant. In another embodiment the implant is an orthopedic implant, wherein the implant is an orthopedic bone void filler, an adjunct to bone fracture stabilization, an intramedullary fixation device, a joint augmentation/replacement device, bone a fixation plate, a screw, a tack, a clip, a staple, a nail, a pin, a rod, an anchor, a screw augmentation device, or a cranial reconstruction device.

The term “hyaluronic acid,” as used herein includes a (co)polymer of acetylglucosamine (C₈H₁₅NO₆) and glucuronic acid (C₆H₁₀O₇) occurring as alternating units.

The term “hyaluronic acid derivative,” as used herein includes hyaluronic acid salts (e.g., sodium, potassium, lithium, ammonium, singly-valent transition metals, and the like, or a combination thereof), hyaluronic acid esters (e.g., alkyl such as methyl, ethyl, n-propyl, isoproypl, n-butyl, isobutyl, sec-butyl, and the like, or a combination thereof), or a combination thereof.

Representative materials for the implant include, but are not limited to, metals and metal alloys (e.g., titanium, titanium alloy, nickel-titanium alloy, tantalum, platinum-iridium alloy, gold, magnesium, stainless steel, chromo-cobalt alloy); ceramics; and biocompatible plastics or polymers (e.g., polyurethanes and/or poly(α-hydroxy ester)s such as polylactides, polyglycolides, polycaprolactones, and the like, and combinations and/or copolymers thereof). Other non-limiting examples of implants include those made from materials disclosed in any of the following U.S. Pat. Nos.: 4,503,157; 4,880,610; 5,047,031; 5,053,212; 5,129,905; 5,164,187; 5,178,845; 5,279,831; 5,336,264; 5,496,399; 5,569,442; 5,571,493; 5,580,623; 5,683,496; 5,683,667; 5,697,981; 5,709,742; 5,782,971; 5,820,632; 5,846,312; 5,885,540; 5,900,254; 5,952,010; 5,962,028; 5,964,932; 5,968,253; 6,002,065; 6,005,162; 6,053,970; 6,334,891; or some combination thereof, the entire contents of which are hereby incorporated by express reference hereto.

In one embodiment, the invention is directed an implant coated with hyaluronic acid or a derivative thereof, wherein the implant comprises metal, a metal alloy, a ceramic, or a combination thereof.

In another embodiment, the invention is directed an implant coated with hyaluronic acid or a derivative thereof, wherein the implant is a metal, a metal alloy, a ceramic, or a combination thereof.

In another embodiment, the invention is directed an implant coated with hyaluronic acid or a derivative thereof, wherein the implant consists essentially of a metal, a metal alloy, a ceramic, or a combination thereof.

When the implant is a ceramic, the ceramic is preferably a calcium-phosphate ceramic, e.g., a calcium phosphate, preferably hydroxyapatite or alternatively tricalcium phosphate. In addition, the body of the implant, may be at least partially filled with material made of calcium sulfate, demineralized bone, autologous bone, or coralline substances. Hydroxyapatite and tricalcium phosphate have the advantage that they become fully integrated into the bone, or are even replaced by new, natural bone tissue.

In another embodiment, the invention is directed an implant coated with hyaluronic acid or a derivative thereof, wherein the implant is substantially free of a polymeric component (i.e., a plastic or polymer). In another embodiment, the amount of polymeric component in the implant is not more than about 25% by weight of polymer and plastic based on the total weight of the implant. In another embodiment, the amount of polymeric component in the implant is not more than about 10% by weight of polymer and plastic based on the total weight of the implant. In another embodiment, the amount of polymeric component in the implant is not more than about 5% by weight of polymer and plastic based on the total weight of the implant.

Non-limiting examples useful implants substantially free of plastic or polymer include a bone void filler, an adjunct to bone fracture stabilization, an intramedullary fixation device, a joint augmentation/replacement device, a bone fixation plate, a screw, a tack, a clip, a staple, a nail, a pin, a rod, an anchor, a scaffold, a stent, a mesh, a sponge, an implant for cell encapsulation, an implant for tissue engineering, a drug delivery device, a bone ingrowth induction catalyst, a monofilament, a multifilament structure, a sheet, a coating, a membrane, a foam, a screw augmentation device, a cranial reconstruction device, a heart valve, or a pacer lead.

The hyaluronic acid or derivative thereof can be obtained from any applicable source, e.g., including, but not limited to, bacterial fermentation; extraction; and/or isolation from animal fluids (e.g., synovial fluid and the like), tissues, bones, or the like. Alternatively, the hyaluronic acid or derivative thereof can be completely or partially chemically synthesized ex vivo. The properties (e.g., molecular weight) of the hyaluronic acid or derivative thereof obtained from different sources may be different. Methods for obtaining hyaluronic acid or a derivative thereof are described in, e.g., U.S. Pat. No. 5,166,331, the entire disclosure of which is expressly incorporated herein be reference.

In one embodiment, the number average molecular weight (e.g., as measured by GPC or SEC against suitable standards such as polyethylene oxide standards) of the hyaluronic acid is at least about 1,000 grams/mole In another embodiment, the number average molecular weight of the hyaluronic acid or derivative thereof is at least about 5,000 g/mol. In another embodiment, the number average molecular weight of the hyaluronic acid or derivative thereof is from about 10,000 grams/mole to about 5,000,000 grams/mole, for example from about 50,000 grams/mole to about 3,000,000 grams/mole, from about 10,000 grams/mole to about 1,000,000 grams/mole, or from about 150,000 grams/mole to about 2,000,000 grams/mole.

In another embodiment, the weight average molecular weight of the hyaluronic acid or derivative thereof (e.g., as measured by GPC or SEC against suitable standards such as polyethylene oxide standards) is at least about 1,500 grams/mole. In another embodiment, the weight average molecular weight of the hyaluronic acid or derivative thereof is at least about 8,000 grams/mole. In another embodiment, the weight average molecular weight of the hyaluronic acid or derivative thereof is from at least about 15,000 grams/mole to about 25,000,000 grams/mole, for example from about 75,000 grams/mole to about 10,000,000 grams/mole, from about 15,000 grams/mole to about 5,000,000 grams/mole, or from about 250,000 grams/mole to about 4,000,000 grams/mole.

In another embodiment, the hyaluronic acid or derivative thereof has a polydispersity (i.e., a ratio of weight average molecular weight to number average molecular weight) from about 1.3 to about 10. In another embodiment, the hyaluronic acid or derivative thereof has a polydispersity from about 1.6 to about 8. In another embodiment, the hyaluronic acid or derivative thereof has a polydispersity from about 1.5 to about 4. In another embodiment, the hyaluronic acid or derivative thereof has a polydispersity from about 2 to about 7. In another embodiment, the hyaluronic acid or derivative thereof has a polydispersity from about 4 to about 9. In another embodiment, the hyaluronic acid or derivative thereof has a polydispersity from about 1.8 to about 2.5.

In another embodiment, the Antimicrobial Coating provides an in vivo resistance to absorption, adhesion, and/or proliferation of a bacteria, such as Staphlococcus aureus or Staphlococcus epidermitis of at least about 5 times better than that exhibited by the implant without the antimicrobial coating. In another embodiment, the in vivo resistance as described above is at least about 10 times better. In another embodiment, in vivo resistance is at least about 100 times better.

Any method capable of forming a coating of a hyaluronic acid or derivative thereof can be utilized to make the coated implants of the invention including, but not limited to dip-coating, application by a brush, spray coating, and any combination thereof. Examples of coating methods can be found in, e.g., U.S. Pat. Nos. 4,500,676, 6,187,369 and 6,106,889 and U.S. Published Application Nos. 2002/0068093 and 2003/0096131, the entire disclosures of which are incorporated herein by express reference hereto. Typically, a composition comprising hyaluronic acid or a derivative thereof and an organic solvent is applied to the implant, and the resultant coated implant is allowed to dry or cure. The Antimicrobial Coating preferably covers at least a majority (i.e., more than 50%) of the surface of the implant); more preferably substantially all of the surface of the implant; most preferably essentially all of the surface of the implant.

In certain embodiments, the surface of the implant material can be modified by chemical and/or physical treatment prior to applying the coating. For example, the implant surface can by physically modified by polishing the surface to reduce surface roughness or abraded to increase surface roughness (e.g., to improve adhesion). Similarly, the surface of the implant can by chemically modified by treating the surface of the implant with, e.g., strong acid or strong base), electropolishing as described in Example 1, anodizing with a metal as described in Example 1, or combinations thereof.

The thickness of the Antimicrobial Coating can be from about 1 micron to about 500 microns. In another embodiment the thickness of the Antimicrobial Coating is from about 3 microns to about 250 microns. In another embodiment, the thickness of the Antimicrobial Coating is from about 5 microns up to about 100 microns.

In another embodiment, the implant further comprises at least a first coat residing on the surface of the implant. Accordingly, in one embodiment, the invention relates to a multi-coated implant comprising: (a) a first coat residing on the surface of the implant; and (b) a second coat comprising hyaluronic acid or a derivative thereof residing on the first coat. Non-limiting examples useful first coats include metals (e.g., titanium, gold, or platinum), ceramic materials (e.g., hydroxyapatite or tricalcium phosphate, or polymers (e.g., an acrylic polymer base coat), or any combination thereof.

The first coat can be the same as, or different from, the implant material. In one embodiment, the composition of the first coat is the same as the composition of the implant. In another embodiment, the composition of the first coat is different from the composition of the implant.

When the implant is coated with a first coat, the composition of the implant can vary. Non-limiting examples of useful implant materials include metals, metal alloys, or ceramics as described above; and/or plastics or polymers, e.g., polyurethanes and/or poly(α-hydroxy ester) such as polylactides, polyglycolides, polycaprolactones, and the like; or any combination thereof.

Methods for coating the implant with a ceramic or polymer include those describe above for coating the implant with hyarluonic acid or a derivative thereof. When the first coat contains a ceramic or polymer, the thickness can range from about 1 micron to about 500 microns; in another embodiment, from about 3 microns to about 250 microns; and in another embodiment, from about 5 microns up to about 100 microns.

In one embodiment, the first coat comprises an acrylic polymer.

In another embodiment, the first coat consists essentially of an acrylic polymer.

In another embodiment, the first coat consists of an acrylic polymer.

Methods for coating an implant material with a metal or metal alloy are described in U.S. Pat. No. 6,428,579, the entire disclosure of which is expressly incorporated herein by reference. Non-limiting examples of metal coats include titanium, gold, silver, and platinum. Preferably the metal first coat, when used, is gold.

When a metal first coat is used, the implant is preferably a titanium or steel implant; more preferably the implant is a titanium implant.

The thickness of the metal coating, when used, is typically from about 10 Angstroms to about 5000 Angstroms. In another embodiment, thickness of the metal coating, when used, is from about 10 Angstroms to about 1000 Angstroms. In another embodiment, thickness of the metal coating, when used, is from about 10 Angstroms to about 250 Angstroms.

It will be understood that the thickness of the first coat, when used, can vary at different points on the surface of the implant. Preferably, the thickness of the first coat is substantially uniform across the entire surface of the implant.

The first coat, when used, preferably covers at least a majority (ie., more than 50%) of the surface of the implant); more preferably substantially all of the surface of the implant; most preferably essentially all of the surface of the implant.

When a first coat is used, the Antimicrobial Coating can have a thickness as described above and can be applied to the first coat by methods described above.

When a first coat is used, the Antimicrobial Coating can further comprise a therapeutic substance as described below.

Optionally, one or more therapeutic substances can be included in the Antimicrobial Coating. The therapeutic substances can include, but are in no way limited to, antibiotics, chemotherapy drugs, growth factors (particularly osteoinductive growth factors) such as bone morphogenetic proteins, endothelial growth factors, insulin growth factors, or the like, or a combination thereof. In one embodiment, the therapeutic substance is added to the Antimicrobial Coating composition. In another embodiment, the therapeutic substance can be complexed with the Antimicrobial Coating composition. In another embodiment, the therapeutic substance can be adhered to the surface of the Antimicrobial Coating. In another embodiment, the therapeutic substance is included as a controlled release formulation within the Antimicrobial Coating composition. Representative therapeutic substances include, but are not limited to, antiseptics (e.g., those antiseptics enumerated in International Publication No. WO 02/082907, broad spectrum biocides, gram-positive antibacterial agents, gram-negative antibacterial agents, guanidium compounds, biguanides, bipyridines, phenoxide antiseptics, alkyl oxides, aryl oxides, thiols, halides, aliphatic amines, aromatic amines, quaternary ammonium-compounds (such as those quaternary ammonium biocides commercially available from BIOSAFE, LLC of Pennsylvania), chemotherapy drugs, growth factors (e.g., osteoinductive growth factors, morphogenetic proteins, endothelial growth factors, insulin growth factors).

Non-limiting examples of useful antimicrobial agents include: Antiamebics, e.g. Arsthinol, Bialamicol, Carbarsone, Cephaeline, Chlorbetamide, Chloroquine, Chlorphenoxamide, Chlortetracycline, Dehydroemetine, Dibromopropamidine, Diloxanide, Diphetarsone, Emetine, Fumagillin, Glaucarubin, Glycobiarsol, 8-Hydroxy-7-iodo-5-quinoline-sulfonic Acid, Iodochlorhydroxyquin, lodoquinol, Paromomycin, Phanquinone, Polybenzarsol, Propamidine, Quinfamide, Scenidazole, Sulfarside, Teclozan, Tetracycline, Thiocarbamizine, Thiocarbarsone, Tinidazole; Antibiotics, e.g. Aminoglycosides (such as Amikacin, Apramycin, Arbekacin, Bambermycins, Butirosin, Dibekacin, Dihydrostreptomycin, Fortimicin(s), Gentamicin, Isepamicin, Kaniamycin, Micronomicin, Neomycin, Neomycin Undecylenate, Netilmicin, Paromomycin, Ribostamycin, Sisomicin, Spectinomycin, Streptomycin, Tobramycin, Trospectomycin), Amphenicols (Azidamfenicol, Chloramphenicol, Florfenicol, Thiamphenicol), Ansamycins (Rifamide, Rifampin, Rifamycin, Rifapentine, Rifaximin), β-Lactams (Carbacephems, Loracarbef, Carbapenems (Biapenem, Imipenem, Meropenem, Panipenem), Cephalosporins (Cefaclor, Cefadroxil, Cefamandole, Cefatrizine, Cefazedone, Cefazolin, Cefcapene Povoxil, Cefclidin, Cefdinir, Cefditoren, Cefepime, Cefetamet, Cefixime, Cefinenoxine, Cefodizime, Cefonicid, Cefoperazone, Ceforanide, Cefotaxime, Cefotiam, Cefozopran, Cefpimizole, Cefpiramide, Cefpirome, Cefpodoxime Proxetil, Cefprozil, Cefroxadine, Cefsulodin, Ceftazidime, Cefteram, Ceftezole, Ceftibuten, Ceftizoxime, Ceftriaxone, Cefuroxime, Cefuzonam, Cephacetrile Sodium, Cephalexin, Cephaloglycin, Cephaloridine, Cephalosporin, Cephalothin, Cephapirin Sodium, Cephradine, Pivcefalexin), Cephamycins (Cefbuperazone, Cefmetazole, Cefminox, Cefotetan, Cefoxitin), Monobactams (Aztreonam, Carumonam, Tigemonam), Oxacephens (Flomoxef, Moxalactam), Penicillins (Amdinocillin, Amdinocillin Pivoxil, Amoxicillin, Ampicillin, Apalcillin, Aspoxicillin, Azidocillin, Azlocillin, Bacampicillin, Benzylpenicillic Acid, Benzylpenicillin Sodium, Carbenicillin, Carindacillin, Clometocillin, Cloxacillin, Cyclacillin, Dicloxacillin, Epicillin, Fenbenicillin, Floxacillin, Hetacillin, Lenampicillin, Metampicillin, Methicillin Sodium, Mezlocillin, Nafcillin Sodium, Oxacillin, Penamecillin, Penethamate Hydriodide, Penicillin G Benethamine, Penicillin G Benzathine, Penicillin G Benzhydrylamine, Penicillin G Calcium, Penicillin G Hydrabamine, Penicillin G Potassium, Penicillin G Procaine, Penicillin N, Penicillin O, Penicillin V, Penicllin V Benzathine, Penicillin V Hydrabamine, Penimepicycline, Phenethicillin Potassium, Piperacillin, Pivampicillin, Propicillin, Quinacillin, Sulbenicillin, Sultamicillin, Talampicillin, Temocillin, Ticarcillin), Ritipenem), Lincosamides (Clindamycin, Lincomycin), Macrolides (Azithromycin, Carbomycin, Clarithromycin, Dirithromycin, Erythromycin, Erythromycin Acistrate, Erythromycin Estolate, Erythromycin Glucoheptonate, Erythromycin Lactobionate, Erythromycin Propionate, Erythromycin Stearate, Josamycin, Leucomycins, Midecamycins, Miokamycin, Oleandomycin, Primycin, Rokitamycin, Rosaramicin, Roxithromycin, Spiramycin, Troleandomycin), Polypeptides (Amphomycin, Bacitracin, Capreomycin, Colistin, Enduracidin, Enviomycin, Fusafungine, Gramicidin S, Gramicidin(s), Mikamycin, Polymyxin, Pristinamycin, Ristocetin, Teicoplanin, Thiostrepton, Tuberactinomycin, Tyrocidine, Tyrothricin, Vancomycin, Viomycin, Virginiamycin, Zinc Bacitracin), Tetracyclines(Apicycline, Chlortetracycline, Clomocycline, Demeclocycline, Doxycycline, Guamecycline, Lymecycline, Meclocycline, Methacycline, Minocycline, Oxytetracycline, Penimepicycline, Pipacycline, Rolitetracycline, Sancycline, Tetracycline), Cycloserine, Mupirocin, Tuberin; synthetic antibacterial agents, e.g. 2,4-Diaminopyrimidines (Brodimoprim, Textroxoprim, Trimethoprim), Nitrofurans (Furaltadone, Furazolium Chloride, Nifuradene, Nifuratel, Nifurfoline, Nifurpirinol, Nifurprazine, Nifurtoinol, Nitrofirantoin), Quinolones and Analogs (Cinoxacin, Ciprofloxacin, Clinafloxacin, Difloxacin, Enoxacin, Fleroxacin, Flumequine, Grepafloxacin, Lomefloxacin, Miloxacin, Nadifloxacin, Nadilixic Acid, Norflaxacin, Ofloxacin, Oxolinic Acid, Pazufloxacin, Pefloxacin, Pipemidic Acid, Piromidic Acid, Rosoxacin, Rufloxacin, Sparfloxacin, Temafloxacin, Tosufloxacin, Trovafloxacin), Sulfonamides (Acetyl Sulfamethoxpyrazine, Benzylsulfamide, Chloramine-B, Chloramine-T, Dichloramine T, N₂-Formylsulfisomidine, N₄-β-D-Glucosylsulfanilamide, Mafenide, 4′-(Methylsulfamoyl)sulfanilanilide, Noprylsulfamide, Phthalylsulfacetamide, Phthalylsulfathiazole, Salazosulfadimidine, Succinylsulfathiazole, Sulfabenzamide, Sulfacetamide, Sulfachlorpyridazine, Sulfachrysoidine, Sulfacytine, Sulfadiazine, Sulfadicramide, Sulfadimethoxine, Sulfadoxine, Sulfaethidole, Sulfaguanidine, Sulfaguanol, Sulfalene, Sulfaloxic, Sulfamerazine, Sulfameter, Sulfamethazine, Sulfamethizole, Sulfamethomidine, Sulfamethoxazole, Sulfamethoxypyridazine, Sulfametrole, Sulfamidochrysoidine, Sulfamoxole, Sulfanilamide, 4-Sulfanilamidosalicylic Acid, N₄-Sulfanilylsulfanilamide, Sulfanilylurea, N-Sulfanilyl-3,4-xylamide, Sulfanitran, Sulfaperine, Sulfaphenazole, Sulfaproxyline, Sulfapyrazine, Sulfapyridine, Sulfasomizole, Sulfasymazine, Sulfathiazole, Sulfathiourea, Sulfatolamide, Sulfisomidine, Sulfisoxazole), Sulfones (Acedapsone, Acediasulfone, Acetosulfone Sodium, Dapsone, Diathymosulfone, Glucosulfone Sodium, Solasulfone, Succisulfone, Sulfanilic Acid, p-Sulfanilylbenzylamine, Sulfoxone Sodium, Thiazolsulfone), Clofoctol, Hexedine, Methenamine, Methenamine Anhydromethylenecitrate, Methenamine Hippurate, Methenamine Mandelate, Methenamine Sulfosalicylate, Nitroxoline, Taurolidine, Xibomol; leprostatic antibacterial agents, such as Acedapsone, Acetosulfone Sodium, Clofazimine, Dapsone, Diathymosulfone, Glucosulfone Sodium, Hydnocarpic Acid, Solasulfone, Succisulfone, Sulfoxone Sodium, antifungal agents, such as Allylamines Butenafine, Naftifine, Terbinafine, Imidazoles ( e.g., Bifonazole, Butoconazole, Cholordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole Nitrate, Sertaconazole, Sulconazole, Tioconazole), Thiocarbamates (Tolcilate, Tolindate, Tolnaftate), Triazoles (Fluconazole, Itraconazole, Saperconazole, Terconazole), Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole Dihydrochloride, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, Zinc Propionate; and the like.

Other antimicrobial agents useful in the present invention include β-lactamase inhibitors (e.g. Clavulanic Acid, Sulbactam, Tazobactam); Chloramphenicols (e.g. Azidamphenicol, Chloramphenicol, Thiaphenicol); Fusidic Acid; synthetic agents such as Trimethoprim, optionally in combination with sulfonamides) and Nitroimidazoles (e.g., Metronidazole, Tinidazole, Nimorazole); Antimycobacterial agents (e.g. Capreomycin, Clofazimine, Dapsone, Ethambutol, Isoniazid, Pyrazinamide, Rifabutin, Rifampicin, Streptomycin, Thioamides); Antiviral agents (e.g. Acryclovir, Amantadine, Azidothymidine, Ganciclovir, Idoxuridine, Tribavirin, Trifluridine, Vidarabine); Interferons (e.g. Interferon α, Interferon β); and antiseptic agents (e.g., Chlorhexidine, Gentian violet, Octenidine, Povidone Iodine, Quaternary ammonium compounds, Silver sulfadiazine, Triclosan).

In one embodiment, the antimicrobial agent is an antibiotic, preferably gentamyicin.

In another embodiment, the antimicrobial agent is an antiseptic, preferably chlorhexidine.

In one embodiment, the invention is directed an implant coated with a coating comprising: (a) hyaluronic acid or a derivative thereof; and (b) and an antimicrobial agent.

In one embodiment, the invention is directed an implant coated with a coating comprising: (a) hyaluronic acid or a derivative thereof; and (b) and an antiseptic agent.

In certain embodiments, the Antimicrobial Coating can comprise one or more polymer additives. Without being limited by theory, Applicants believe that the addition of a polymer, e.g., an elastic film forming polymer, can improve the structural characteristics of the Antimicrobial Coating such as improved flexibility, adhesion and/or as resistance to cracking . Any polymer can be used provided the polymer is biocompatible and does not significantly interfere with the desired characteristics of the hyaluronic acid component. Typically, the polymer, when used, is bioadsorbable or erodible. More preferably, the polymer, when used, is bioadsorbable. A non-limiting examples of a useful polymers include polyurethane (see U.S. Pat. No. 4,500,676, the entire disclosure of which is incorporated herein be reference); polylactides; polyglycolides; homopolymers or copolymers of monomers selected from the group consisting of L-lactide; L-lactic acid; D-lactide; D-lactic acid; D,L-lactide; glycolide; α-hydroxybutyric acid; α-hydroxyvaleric acid; α-hydroxyacetic acid; α-hydroxycaproic acid; α-hydroxyheptanoic acid; α-hydroxydecanoic acid; α-hydroxymyristic acid; α-hydroxyoctanoic acid; α-hydroxystearic acid; hydroxybutyrate; hydroxyvalerate; β-propiolactide; β-propiolactic acid; γ-caprolactone; β-caprolactone; γ-butyrolactone; pivalolactone; tetramethylglycolide; tetramethylglycolic acid; dimethylglycolic acid; trimethylene carbonate; dioxanone; those monomers that form liquid crystal (co)polymers; those monomers that form cellulose; those monomers that form cellulose acetate; those monomers that form carboxymethylcellulose; those monomers that form hydroxypropylmethyl-cellulose; polyurethane precursors comprising macrodiols selected from the group consisting of polycaprolactone, poly(ethylene oxide), poly(ethylene glycol), poly(ethylene adipate), poly(butylene oxide), and a mixture thereof, isocyanate-functional compounds selected from the group consisting of hexamethylene diisocyanate, isophorone diisocyanate, cyclohexane diisocyanate, hydrogenated methylene diphenylene diisocyanate, and a mixture thereof, and chain extenders selected from the group consisting of ethylenediamine, 1,4-butanediol, 1,2-butanediol, 2-amino-1-butanol, thiodiethylene diol, 2-mercaptoethyl ether, 3-hexyne-2,5-diol, citric acid, and a mixture thereof; collagen, alginates (e.g., sodium or calcium alginate), polysaccarides such as chitin and chitosan, poly(propylene fumarate); and any mixture thereof.

In one embodiment, the Antimicrobial Coating further comprises at least one or more elastic film-forming polymer additives.

In another embodiment, the Antimicrobial Coating comprises hyaluronic acid.

In another embodiment, the Antimicrobial Coating comprises sodium hyaluronate.

In another embodiment, the Antimicrobial Coating consists essentially of hyaluronic acid, sodium hyaluronate, or a combination thereof.

In another embodiment, the Antimicrobial Coating consists essentially of hyaluronic acid or a derivative thereof.

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

Preparation of the Titanium Substrates: Unalloyed titanium discs, 12.7 mm diameter×1.0 mm thick, were electrochemically anodized and dip coated with an acrylic polymer base coat and a hyaluronan top coat. All samples containing hyaluronic acid were sterilized by ethylene oxide.

When the coating comprise chlorhexidine, the substrates containing hyaluronic acid were dip-coated in an aqueous solution of 1.5% chlorhexidine diacetate antiseptic.

### Example 1

Example 1 describes the results of microbial testing on different titanium surfaces (substrates) that have been coated with hyaluronic acid.

Substrates used in the study: Table 1 lists the various substrates used in the study.

TABLE 1

| Label | Description |
|---|---|
| TSS | Unalloyed Ti, gold anodized, Synthes (USA) |
| TS | Unalloyed Ti, gold anodized, Synthes (USA) |
| TLF | Low friction grey anodized Ti, Synthes (USA) |
| TIG | Nitrogen ion implanted TSS |
| THY | TSS grafted with sodium hyaluronate |
| TAST | TSS with polymer cell promotion |
| TC | Chemically polished Ti, gold anodized, Synthes (USA) |
| TE | Electropolished Ti, gold anodized, Synthes (USA) |
| TM | Mechanically polished Ti, gold anodized, Synthes (USA) |

The TSS samples (Synthes (USA), Paoli, Pa.) were made out of implant quality titanium grade 4, meeting ASTM F67 implant material specification, cut from bar, deburred, tumbled with ceramics, cleaned and gold anodized (oxidized) as described in Injury 26(S1):21-27 (1995), then coated with various surfaces treatments as described above, except for sample TSS which was not coated.

The TS samples (Synthes (USA)) were also made out of implant quality titanium grade 4, meeting ASTM F67 implant material specification, punched from sheet (TS) or cut from bar, deburred, tumbled with ceramics, and cleaned. The TS samples was gold anodized (oxidized) to provide a surface layer of titanium oxide. The TC, TE and TM surfaces were polished using one of the methods below, before being gold anodized. The electropolished surfaces were produced by immersing the samples in a liquid (electrolyte) and applying an electric current. The chemical polishing was accomplished by immersing the samples in a liquid chemical without applying an electric current. Finally the mechanically polished surfaces were produced using diamond paste on the sample surfaces.

For the TIG surface, the nitrogen implantation was only applied to one side causing a change in the optical properties of the anodized film.

TLF surfaces were not gold anodized.

The surface topography of each sample was quantitatively measured by laser profilometry (UBM Messtechnik GmbH, Germany). The surfaces were also imaged with a Hitachi S-4700 scanning electron microscope (SEM), using the secondary electron (SE) detection mode at an acceleration voltage of about 4 kV and an emission current of about 40 μA. The Rₘ and Rᵣₘₛ roughness parameters (see Sittig et al., J. Mater. Sci. Mater. Med. 10:35-36 (1999) for each surface were determined and are shown in Table 2 below. Differences in roughness were observed between the samples, with TS, THY, TIG, TLF and TAST showing comparable roughness, TSS and TC being smoother, and TE and TM being the smoothest. The results of the surface roughness study are provided in Table 2.

TABLE 2

| | Test Surface | | | | | | | | |
|---|---|---|---|---|---|---|---|---|---|
| | TS | TSS | THY | TIG | TLF | TAST | TC | TE | TM |
| Rₘ | 1.15 | 0.83 | 1.09 | 1.05 | 1.14 | 1.09 | 0.67 | 0.18 | 0.15 |
| Rᵣₘₛ | 1.45 | 1.08 | 1.35 | 1.31 | 1.42 | 1.46 | 0.85 | 0.23 | 0.2 |

Rₘ represents the arithmetic mean and Rᵣₘₛ represents the root mean square.

S. aureus 8325-4 was grown in Brain Heart Infusion broth (BHI) to an OD₆₀₀ of about 1 at approximately 37° C. in a shaker bath and was used to inoculate 1 mL of pre-warmed BHI in 4 well plates containing one of the test surfaces described in Table 1 to a starting OD₆₀₀ of about 0.05. Each test sample was incubated without shaking at about 37° C. for 1 h. To visualize S. aureus adherence to the TS, TSS, THY, TIG, TLF, and TAST surfaces with an SEM, adherent bacteria were fixed with glutaraldehyde, post-stained with about 1% OSO₄, dehydrated, critical point dried, coated with Au/Pd, and visualized with an SEM using a backscattered electron (BSE) detector (see Richards et al., J. Microsc. 177: 43-52 (1995)) at an acceleration voltage of about 5 kV and emission current of about 40 μA. To quantify the density of S. aureus adhering, bacteria were stained with a fluorescent redox dye, 5-cyano,2-ditolyl tetrazolium chloride (CTC) (see An et al., J. Microbiol. Methods 24: 29 (1995)) for about 1 h and visualized with a Zeiss Axioplan 2 Epifluorescence microscope fitted with a Axiocam camera. The density of live bacteria adhering to the surface observed in each image was counted using KS400 software, and analyzed statistically using a one-way ANOVA with Tukey test.

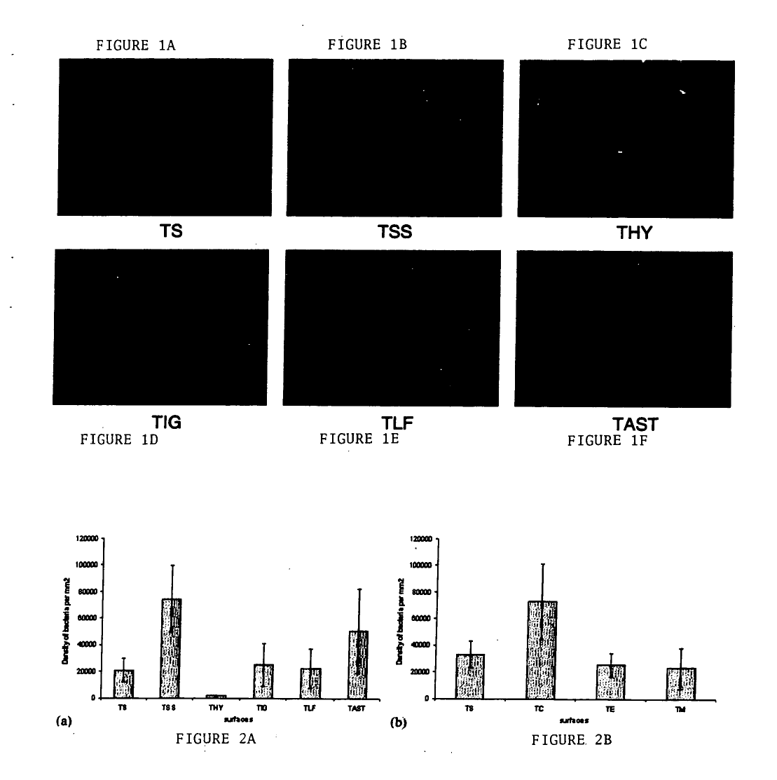

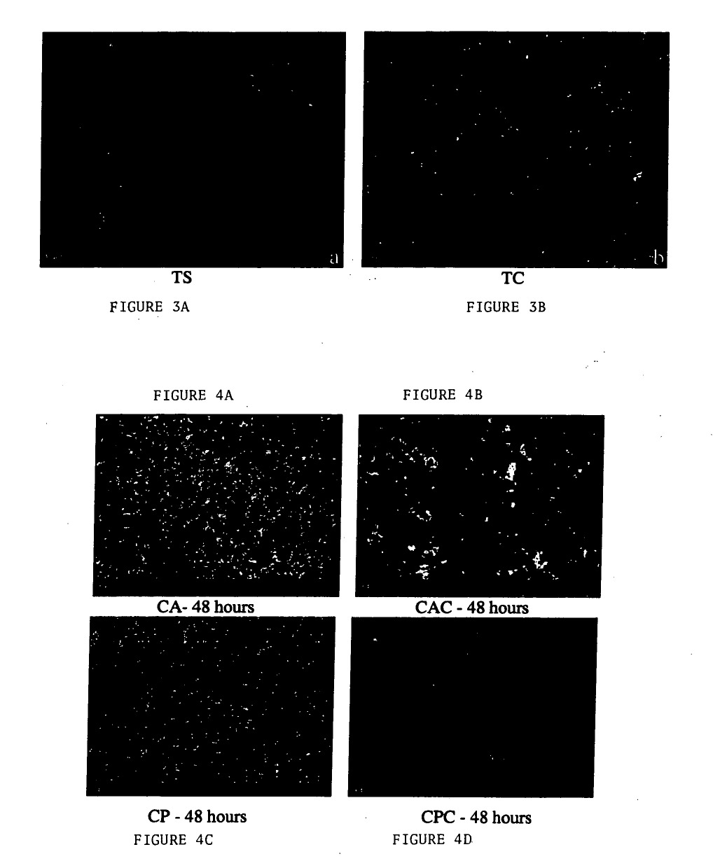

SEM images of the coated surfaces showed that S. aureus adhered to all of the surfaces prepared (FIG. 1), with the exception of the THY surface (FIG. 1) (i.e., the surface containing sodium hyaluronate). (SEM images of the surface topographies also confirmed the roughness parameter results (see FIG. 1 where the surfaces can be seen behind the bacteria)). Fluorescence microscopy confirmed the SEM imaging. Significantly less S. aureus was counted on the THY surface in comparison to the other coated surfaces (FIG. 2a). The amount of adhesion was highest for the TSS and TAST surfaces. Significantly more S. aureus adhered to the TC surface than to the TS (control) or other polished titanium surfaces (TE and TM) as determined using fluorescence microscopy images (FIG. 3). The density of bacteria on TS, TE and TM were comparable (FIG. 2b), despite differences in surface roughness (Table 2). With the exception of THY, no major differences were observed in S. aureus adhesion to the different coated samples. On the THY surface used in this study (FIG. 1), the density of S. aureus was minimal compared to TS and TSS (FIG. 1), the control surfaces, thus suggesting that a THY coating is useful for inhibiting bacterial adhesion to metal and polymer implants.

The results of this in vitro study indicate that polishing or coating the surfaces alone did not have a significant effect on minimizing S. aureus adhesion to these surfaces. The study confirmed that the TAST surface could promote bacterial adhesion, as well as the cell adhesion it is designed to promote. In contrast, coating titanium (TSS) with sodium hyaluronate, significantly decreased the density of S. aureus adhering to the surfaces.

### Example 2

Example 2 shows that a coating comprising a polymer, hyaluronic acid and an antimicrobial agent (e.g., chlorhexidine) is useful for preventing microbial growth on a gold anodized titanium substrate.

Gold anodized titanium (Synthes (USA)) was dipcoated as described above with various combinations of hyaluronic acid, chlorhexidine and/or polymer. The various coatings are provided in Table 3.

TABLE 3

| Group | Code | Surface Coating | Source | Adhesion Exp. |
|---|---|---|---|---|
| 1 | CA | HA/amorphous | Mathys | Bacteria + Cell |
| | | polycarbonate | | |
| 1 | CA + C | HA/amorphous | Mathys | Bacteria + Cell |
| | | polycarbonate + | | |
| | | chlorhexidine | | |
| 1 | CP | 70/30 | ARI | Bacteria + Cell |
| | | polyurethane | | |
| 1 | CP + C | 70/30 | ARI | Bacteria + Cell |
| | | polyurethane + | | |
| | | chlorhexidine | | |
| 2 | CH | Hyaluronic acid | Synthes (Biocoat) | Bacteria + Cell |
| 2 | CH + C | Hyaluronic acid + | Synthes (Biocoat) | Bacteria + Cell |
| | | chlorhexidine | | |
| 2 | CHP | Hyaluronic acid- | Synthes (Biocoat) | Bacteria only |
| | | Cecropin | | |
| 2 | CHP10 | Hyaluronic acid- | Synthes (Biocoat) | Bacteria only |
| | | Cecropin 1/10 | | |
| | | dilution | | |
| 2 | CHR | Hyaluronic | Synthes (Biocoat) | Bacteria only |
| | | acid-RGD | | |
| 2 | CHR10 | Hyaluronic | Synthes (Biocoat) | Bacteria only |
| | | acid-RGD 1/10 | | |
| | | dilution | | |

Surface characterization of the metal base substrates. Surface roughness measurements were carried on the coated substrates as described in Example 1, and the results are provided in Table 4.

TABLE 4

| Substrate | CA | CP | CH | CpTi* |
|---|---|---|---|---|
| Ra, μm** | 0.43 | 0.86 | 0.86 | 0.66 |

*Super-pure titanium.
**Ra is the arithmetic average of the absolute values of all points of a measurement profile (see Sittig et al., J. Mater. Sci. Mater. Med. 10: 35-36 (1999

Fixation for SEM All chemicals were purchased from Fluka Chemie AG (Buchs, Switzerland) unless otherwise stated. All procedures were carried out at 22-25° C., and piperazine-N′N′-bis-2-ethane sulphonic acid (PIPES) buffer was used at a concentration of 0.1 molar and a pH of 7.4 unless otherwise stated. Initially the cells or bacteria were rinsed for 2 minutes in PIPES buffer before being fixed in 2.5% glutaraldehyde in PIPES for 5 minutes. The cells/bacteria were rinsed three times for 2 minutes in PIPES buffer, and post-fixed with 1% osmium tetroxide (Simec Trade AG, Zofingen, Switzerland) in PIPES buffer, pH 6.8, for 60 minutes. The cells/bacteria were then rinsed three times in double distilled water, for two minutes each wash before dehydration through an ethanol series (50%, 70%, 96% and 100%) for 5 minutes each wash. The ethanol was then substituted using 1:3, 1:1 and 3:1 1,2-trichlorotrifluorethane:ethanol, then 100% (v/v) 1,2-trichlorotrifluorethane. Following this the samples were critically point dried in a POLARON E3000 critical point drier (Agar Scientific, Stansted, UK), and coated with 10 nm of gold/palladium (80/20) using a Baltec MED 020 unit (Baltec, Buchs, Liechtenstein). Specimens were examined using a Hitachi S-4700 FESEM, operated in HC-BSE detection mode. 10 images were taken from randomly chosen co-ordinates on the surfaces.

Fibroblast cell culturing. Infinity™ Telomerase-immortalized primary human fibroblasts (hTERT-BJ1) stock cultures were recovered from liquid nitrogen and plated at 300,000 cells per 25 cm² plastic flask in Dulbecco's modified Eagle's medium (DMEM) with 10% foetal calf serum (FCS), Medium 199, 200 mM L-glutamine, and 100 mM sodium pyruvate (no antibiotics). After 2-3 days hTERT cells were detached with 0.25% trypsin and 0.02% ethylenediamine tetra-acetic acid (EDTA), disodium salt (calcium and magnesium free) in tyrode buffered saline solution (TBSS). Recovered cells were rinsed and cultured at an inoculum of 10,000 cells per well in DMEM with 10% FCS (as above) on the different surfaces for 48 hours and 96 hours, with media change every 24 h, before fixation for SEM study.

Results of the above-described studies are discussed below:

S. epidermidis adhesion/growth: SEM analyses of the substrates contacted with S. epidermis exhibited bacteria all over the surfaces without chlorhexidine, while few were seen on the surfaces with chlorhexidine (FIG. 4). Bubble-like structures were seen on CAC. Plate counts showed that S. epidermidis recovered from the surfaces without chlorhexidine were viable, while those recovered from the surfaces with chlorhexidine were not viable in the early time points, but were more viable by 96 h. The results suggest that S. epidermidis confers resistance to chlorhexidine or that the chlorhexidine concentrations were so diminished by 96 h, any bacteria present in the media and surface were able to flourish.

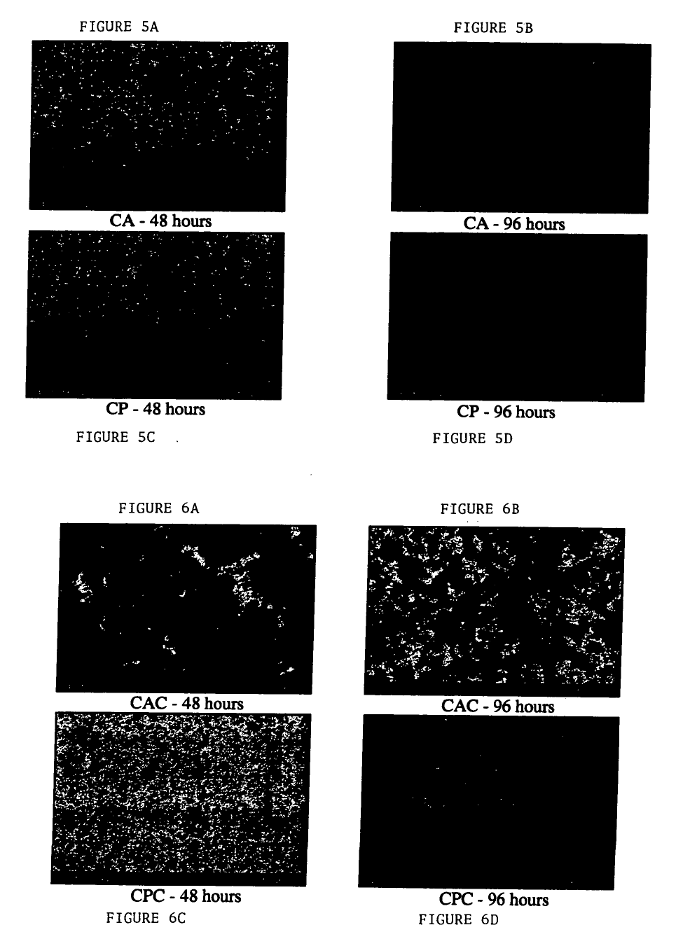

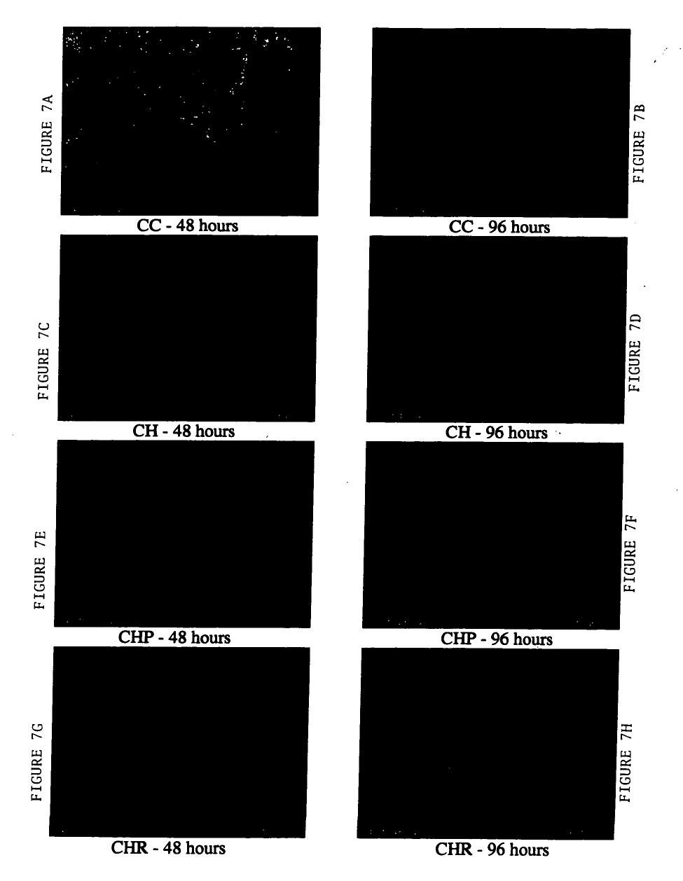

hTERT Fibroblast Adhesion: hTERT fibroblast adhesion studies were carried out for the Group 1 and Group II surfaces (see Table 2). After 48 h and 96 h of culturing, well-spread cells were observed on Group 1 surfaces without chlorhexidine (FIG. 5), while no intact cells were seen on the surfaces containing chlorhexidine (FIG. 6). For the Group 2 surfaces, few spread cells were found on the CHP and CHR surfaces (FIG. 7). No adherent cells were found on CH surface (hyaluronic acid) (FIG. 7) or those surfaces containing chlorhexidine (FIG. 7).

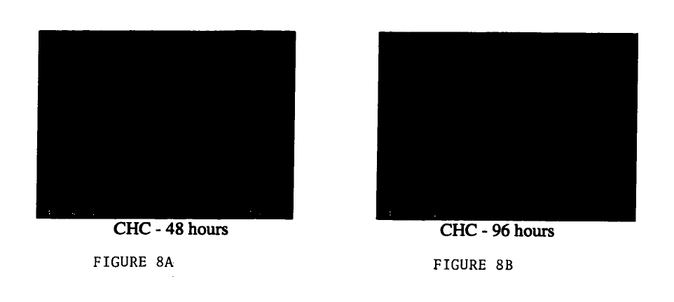

Some of the single cells studied in the hTERT fibroblast adhesion studies were imaged and the amount of spreading analysed using image analysis. The results for CHC (FIG. 8) showed few spread cells on the surface after 96 h.

A separate experiment was carried out to measure the cytotoxicity of different chlorhexidine concentrations on the viability/adhesion of hTERT cells. hTERT cells were cultured as described above onto Thermanox discs in four well plates, but the DMEM with 10% FCS was inoculated with 0.1%, 1% or 10% chlorhexidine. Two control discs were also included containing just DMEM with 10% FCS, one disc was in the same plate as the chlorhexidine samples and the other in a separate four well plate. Plates were incubated at 37° C. for 24 h, before the media was removed and 1 ml DMEM (no FCS) containing 1 μl/ml Calcein AM (reacts with live cells) and 1 μl/ml Ethidium homodimer (reacts with dead cells) was added. The plates were incubated at 37° C. in the dark for a further 30 minutes, then the Thermanox discs were imaged using a Zeiss Epifluorescence microscope. Live well spread cells were seen on the sample not exposed to chlorhexidine, while dead cells were observed on the surface cultured in the same plate as samples exposed to chlorhexidine. No cells were seen on the samples exposed to 0.1% or 1% chlorhexidine. The result suggests that even a low level of chlorhexidine can be used to kill fibroblast cells.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference.

1. A coated implant, wherein the coating comprises and the implant comprises a metal, a metal alloy, or a ceramic.

2. The coated implant of claim 1, wherein the coating reduces at least one of absorption, adhesion, or proliferation of a bacteria by a factor of at least about 5 times better compared to an implant without the coating.

3. The coated implant of claim 2, wherein the bacteria is Staphlococcus aureus, Staphlococcus epidermidis, or a mixture thereof.

4. The coated implant of claim 3, wherein the bacteria is Staphlococcus aureus.

5. The coated implant of claim 1, in the form of a void filler, an adjunct to bone fracture stabilization, an intramedullary fixation device, a joint augmentation/replacement device, a bone fixation plate, a screw, a tack, a clip, a staple, a nail, a pin, a rod, an anchor, a scaffold, a stent, a mesh, a sponge, an implant for cell encapsulation, an implant for tissue engineering, a drug delivery device, a bone ingrowth induction catalyst, a monofilament, a multifilament structure, a sheet, a coating, a membrane, a foam, a screw augmentation device, a cranial reconstruction device, a heart valve, or a pacer lead.

6. The coated implant of claim 1, wherein the thickness of the coating is from about 1 microns to about 500 microns.

7. The coated implant of claim 6, wherein the thickness of the coating is from about 3 microns to about 250 microns.

8. The coated implant of claim 2, wherein the coating reduces at least one of absorption, adhesion, or proliferation of Staphlococcus aureus by a factor of at least about 10 times.

9. The coated implant of claim 2, wherein the coating reduces at least one of absorption, adhesion, or proliferation of Staphlococcus aureus by a factor of at least about 100 times.

10. The coated implant of claim 1, wherein the antimicrobial coating comprises hyaluronic acid.

11. The coated implant of claim 1, wherein the antimicrobial coating comprises sodium hyaluronate.

12. The coated implant of claim 1, wherein the antimicrobial coating consists essentially of hyaluronic acid, sodium hyaluronate, or a combination thereof.

13. The coated implant of claim 1, wherein the antimicrobial coating further comprises a therapeutic substance.

14. The coated implant of claim 13, wherein the therapeutic substance comprises an antibiotic.

15. The coated implant of claim 11, wherein the implant is substantially free of a polymeric component.

16. A coated orthopedic implant, wherein the coating comprises hyaluronic acid or a derivative thereof.

17. The coated orthopedic implant of claim 16, wherein the orthopedic implant is an orthopedic bone void filler, an adjunct to bone fracture stabilization, an intramedullary fixation device, a joint augmentation/replacement device, bone a fixation plate, a screw, a tack, a clip, a staple, a nail, a pin, a rod, an anchor, a screw augmentation device, or a cranial reconstruction device.

18. The coated orthopedic implant of claim 16, wherein the thickness of the coating is from about 1 microns to about 500 microns.

19. The coated orthopedic implant of claim 18, wherein the thickness of the coating is from about 3 microns to about 250 microns.

20. The coated orthopedic implant of claim 16, wherein the antimicrobial coating comprises hyaluronic acid.

21. The coated orthopedic implant of claim 16, wherein the antimicrobial coating comprises sodium hyaluronate.

22. The coated orthopedic implant of claim 16, wherein the antimicrobial coating consists essentially of hyaluronic acid, sodium hyaluronate, or a combination thereof.

23. The coated orthopedic implant of claim 16, wherein the antimicrobial coating further comprises a therapeutic substance.

24. The coated orthopedic implant of claim 23, wherein the therapeutic substance comprises an antibiotic.

25. A multi-coated implant comprising: (a) a first layer comprising a first coat residing on the surface of the implant; and (b) a second coat comprising hyaluronic acid or a derivative thereof residing on the first layer.

26. The multi-coated implant of claim 25, wherein the first layer comprises a metal, a metal alloy, a ceramic, or a polymer.

27. The multi-coated implant of claim 26, wherein the first layer has a thickness from about 10 Angstroms to about 5000 Angstroms.

28. The multi-coated implant of claim 27, wherein first layer has a thickness of the coating is from about 10 Angstroms to about 1000 Angstroms.

29. The multi-coated implant of claim 25, wherein the second coat comprises hyaluronic acid.

30. The multi-coated implant of claim 25, wherein the second coat comprises sodium hyaluronate.

31. The multi-coated implant of claim 25, wherein the second coat consists essentially of hyaluronic acid, sodium hyaluronate, or a combination thereof.

32. The multi-coated implant of claim 25, wherein the second coat further comprises a therapeutic substance.

33. The multi-coated implant of claim 32, wherein the therapeutic substance comprises an antibiotic or an antiseptic.

34. A coated implant, wherein the coating consists essentially of hyaluronic acid or a derivative thereof; and the implant is a void filler, an adjunct to bone fracture stabilization, an intramedullary fixation device, a joint augmentation/replacement device, a bone fixation plate, a screw, a tack, a clip, a staple, a nail, a pin, a rod, an anchor, a scaffold, a stent, a mesh, a sponge, an implant for cell encapsulation, an implant for tissue engineering, a drug delivery device, a bone ingrowth induction catalyst, a monofilament, a multifilament structure, a sheet, a coating, a membrane, a foam, a screw augmentation device, a cranial reconstruction device, a heart valve or a pacer lead.

35. A method for making a coated implant comprising:
(a) providing and implant comprising a metal, a metal alloy, or a ceramic; and
(b) coating the implant with a second coat comprising hyaluronic acid or a derivative thereof.

36. The method of claim 35, wherein the implant further comprises a first coat.

37. The method of claim 36, wherein the first coat comprises a metal, a metal alloy, a ceramic, or a polymer.

38. The method of claim 37, wherein the first coat comprises an acrylic polymer.
